(19) United States
(12) Patent Application Publication
(10) Pub. No.: US 2016/0031771 A1
(43) Pub. Date: Feb. 4, 2016

(54) MFI Aluminosilicate Molecular Sieves and Methods for Using Same for Xylene Isomerization

(71) Applicants: Philip Owen NUBEL, (US); Jeffrey Allen AMELSE, (US)

(72) Inventors: Philip O. Nubel, Naperville, IL (US); Jeffrey A. Amelse, Batavia, IL (US)

(73) Assignee: BP Corporation North America Inc., Naperville, IL (US)

(21) Appl. No.: 14/777,057

(22) PCT Filed: Mar. 12, 2014
(86) PCT No.: PCT/US2014/024438

§ 371 Date: Sep. 15, 2015

Related U.S. Application Data
(60) Provisional application No. 61/793,180, filed on Mar. 15, 2013.

(51) Int. Cl. C07C 5/27; C07C 5/13; C07C 5/10; B01J 29/40

(52) U.S. Cl. CPC C07C 5/2737; B01J 29/40; C07C 5/13; C07C 5/10; C07C 5/277; C07C 2529/40

(57) ABSTRACT

MFI aluminosilicate molecular sieve catalysts are prepared from tetra-functional orthosilicates [e.g., Si(OR₁)(OR₂)(OR₃)(OR₄), wherein R₁R₂R₃R₄ is each independently a C₁₋₁₀alkyl or aryl.] as the silicon source. Such catalysts are useful for hydrocarbon conversion reactions including isomerization of xylenes in C₈ aromatics feed stocks to produce p-xylene. Advantageously, it has been found that the MFI aluminosilicate molecular sieve catalysts of the invention are more selective than conventional commercial MFI catalysts, resulting in reduced formation of transmethylation byproducts (C₇ and C₉ aromatics) while simultaneously providing a high degree of xylene isomerization.

### FIELD OF THE INVENTION

The disclosure relates to methods for making and using an isomerization catalyst, and in particular, methods for making and using MFI aluminosilicate molecular sieves prepared using tetra-functional-orthosilicate precursors, and catalyst systems and isomerization reactors containing the same in xylene isomerization.

### BACKGROUND

Xylene isomerization is an important chemical process. P-xylene is useful in the manufacture of terephthalic acid which is an intermediate in the manufacture of polyesters. Typically p-xylene is derived from mixtures of C₈ aromatics separated from such raw materials as petroleum reformates, usually by distillation. The C₈ aromatics in such mixtures are ethylbenzene, p-xylene, m-xylene, and o-xylene.

Xylene isomerization catalysts can be classified into three types based upon the manner in which they convert ethylbenzene: (1) naphthene pool catalysts, (2) transalkylation catalysts, and (3) hydrodeethylation catalyst. Naphthene pool catalysts, containing a strong hydrogenation function (e.g, platinum) and an acid function (e.g., a molecular sieve) can convert a portion of the ethylbenzene to xylenes via naphthene intermediates. Transalkylation catalysts generally contain a shape selective molecular sieve which inhibits certain reactions based on the size of the reactants, products, and/or intermediates involved. For example, the pores can allow ethyl transfer to occur via a dealkylation/realkylation mechanism, but can inhibit methyl transfer which requires the formation of a bulky biphenylalkane intermediate. Finally, hydrodeethylation catalysts, containing an acidic shape-selective catalyst and an ethylene-selective hydrogenation catalyst component, can convert ethylbenzene to benzene and ethane via an ethylene intermediate. However, such catalysts often sacrifice xylene isomerization efficiency to efficiently remove ethylbenzene.

In contrast, dual bed catalyst systems can more efficiently convert ethylbenzene and non-aromatics in mixed C₈ aromatic feeds, while simultaneously converting xylenes to thermal equilibrium. Dual bed xylene isomerization catalysts consist of an ethylbenzene conversion catalyst component and a xylene isomerization component. Typically, the ethylbenzene conversion catalyst is selective for converting ethylbenzene to products which can be separated via distillation, though it is a less effective xylene isomerization catalyst; that is, it does not produce an equilibrium distribution of xylene isomers. This catalyst system has an advantage over a conventional single bed xylene isomerization catalyst in that it affords lower xylene losses. However, in order to maximize p-xylene yields from dual bed catalyst systems, the xylene isomerization component should demonstrate high xylene isomerization activity, but low xylene lossactivity to prevent degradation of catalytic selectivity.

MFI aluminosilicate molecular sieves are employed commercially for hydrocarbon conversion reactions including isomerization of xylenes in xylene isomers and C₈ aromatics to produce p-xylene. Commercial MFI aluminosilicate molecular, however, also typically catalyze transalkylation side-reactions, and in particular transmethylation reactions of xylenes that reduce the yield of p-xylene product. For example, typical MFI aluminosilicate molecular sieves cause some degree of xylene-xylene transmethylation and xylene-ethylbenzene transmethylation, resulting in undesirable conversion of xylenes to C₇ and C₉ products. In addition, typical commercial MFI aluminosilicate molecular sieves have difficulty achieving a high degree of xylene isomerization such that the product xylene mixture is below thermodynamic equilibrium. Thus, there continues to be a need for improved xylene isomerization catalysts that can maximize yields of p-xylene while minimizing xylene loss to transmethylation reactions.

### BRIEF SUMMARY OF THE INVENTION

The present invention provides MFI aluminosilicate molecular sieves having unexpectedly high xylene isomerization activity while simultaneously yielding less transmethylation byproducts (C₇ and C₉ aromatics) compared to industry standard catalysts. Also provided are methods for use of these MFI aluminosilicate molecular sieves for enriching the p-xylene content of a hydrocarbon-containing feed stream comprising xylene isomers. Such catalysts include MFI aluminosilicate molecular sieves that can be prepared, for example, from tetra-functional orthosilicate precursors, such as tetraethylorthosilicate.

Accordingly, in one aspect, the invention provides methods for increasing the proportion of p-xylene (pX) in a hydrocarbon-containing feed stream comprising xylene isomers. The method includes contacting the hydrocarbon-containing feed stream with an isomerization catalyst under conditions suitable to yield a stream enriched in p-xylene with respect to the hydrocarbon-containing feed stream, in which the isomerization catalyst includes a MFI aluminosilicate molecular sieves prepared using a silicon source including, for example, a compound of the formula, Si(OR₁)(OR₂)(OR₃)(OR₄), wherein R₁R₂R₃R₄ is each independently C₁₋₁₀alkyl or aryl.

In another aspect, the invention provides methods for increasing the proportion of p-xylene (pX) in a hydrocarbon-containing feed stream comprising xylene isomers, said method comprising: contacting the hydrocarbon-containing feed stream with an isomerization catalyst under conditions suitable to yield a stream enriched in p-xylene with respect to the hydrocarbon-containing feed stream, in which the isomerization catalyst includes a MFI aluminosilicate molecular sieve; and the pX enriched stream contains at least 23.5 wt. % pX/X, where pX/X is the ratio of p-xylene to total xylenes in the stream, as defined below, and less than 1.5 wt. % net toluene byproduct.

In yet another aspect, the invention provides methods for increasing the proportion of p-xylene (pX) in a hydrocarbon-containing feed stream including xylene isomers, said method including: contacting the hydrocarbon-containing feed stream with an isomerization catalyst under conditions suitable to yield a stream enriched in p-xylene with respect to the hydrocarbon-containing feed stream, in which the isomerization catalyst includes a MFI aluminosilicate molecular sieve; and the pX enriched stream contains at least 23.8 wt. % pX/X and less than 0.6 wt. % net trimethylbenzene byproduct.

In another aspect, the invention provides methods for increasing the proportion of p-xylene (pX) in a hydrocarbon-containing feed stream including xylene isomers, said method including: contacting the hydrocarbon-containing feed stream with an isomerization catalyst under conditions suitable to yield a stream enriched in p-xylene with respect to the hydrocarbon-containing feed stream, in which the isomerization catalyst includes a MFI aluminosilicate molecular sieve; and the pX enriched stream contains at least 23.5 wt. % pX/X and a ratio of pX/X to the sum of net wt. % trimethylbenzene byproduct and net wt. % toluene byproduct of greater than 4.0.

In another aspect, the invention provides catalyst systems for enriching a xylene isomers feed in p-xylene including a first bed including an ethylbenzene (EB) conversion catalyst and a second bed including an isomerization catalyst that is a MFI aluminosilicate catalyst, such as a MFI aluminosilicate molecular sieve prepared using a silicon source that includes a compound of the formula, Si(OR)₄, wherein R is C₁₋₁₀alkyl or aryl.

In another aspect, the invention provides a xylene isomerization reactor having a reaction zone containing a catalyst system as described above.

### BRIEF DESCRIPTION OF THE DRAWINGS

FIG. 1a is a flow diagram illustrating one illustrative embodiment of a method for xylene isomerization.

FIG. 1b is a flow diagram illustrating another illustrative embodiment of a method for xylene isomerization.

FIG. 1c is a flow diagram illustrating a third illustrative embodiment of a method for xylene isomerization.

FIG. 2 shows SEM Images of a MFI aluminosilicate molecular sieve prepared from TEOS (containing 1.5 wt. % Al and 99% crystalline by XRD).

FIG. 3 is a plot of net yield of toluene vs. % pX/xylenes (30-52% EB conversion data) for various molecular sieve catalysts.

FIG. 4 a plot of net yield of trimethylbenzene vs. % pX/xylenes for various molecular sieve catalysts.

FIG. 5 is a plot of net yield pX/net yield (toluene+trimethylbenzene) vs. % pX/xylenes for various molecular sieve catalysts.

### DETAILED DESCRIPTION OF PREFERRED EMBODIMENTS

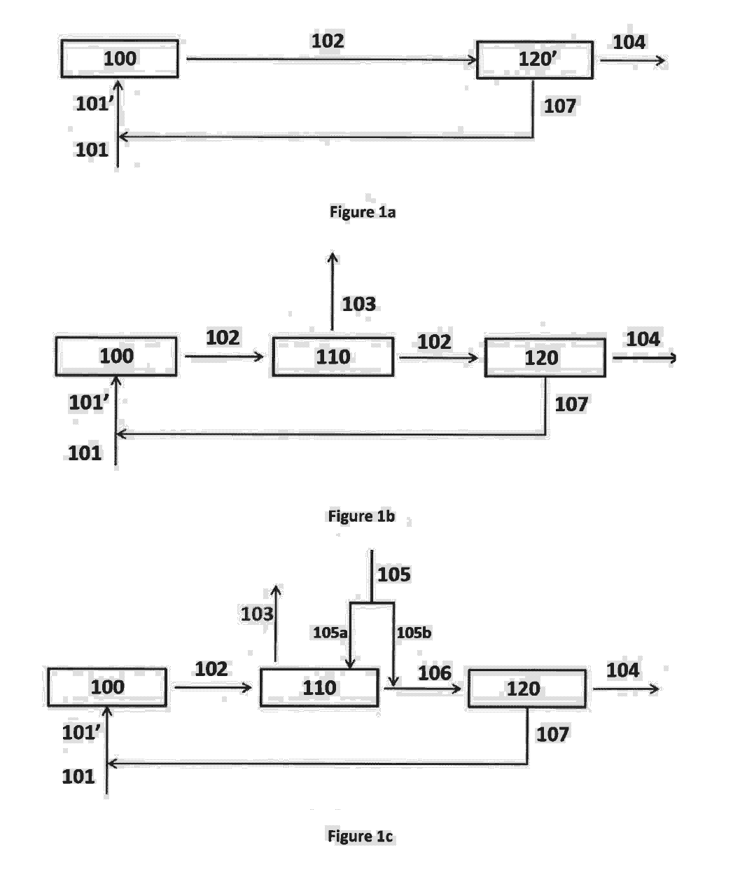

In a first aspect, the invention provides methods for increasing the proportion of p-xylene (pX) in a hydrocarbon-containing feed stream including xylene isomers. The method includes, referring to FIG. 1a, contacting in a reaction zone of a reactor (100) a hydrocarbon-containing feed stream (101 or 101′) with an isomerization catalyst of the application under conditions suitable to yield a stream enriched in p-xylene (102) with respect to the hydrocarbon-containing feed stream, where the isomerization catalyst includes a boroaluminosilicate molecular sieve. The pX enriched stream (102), can generally contain benzene, toluene, and xylene isomers (i.e., ethylbenzene (EB), o-xylene (oX), m-xylene (mX) and p-xylene (pX)). The methods may be carried out as batch, semi-continuous, or continuous operations.

In certain embodiments, the hydrocarbon-containing feed stream includes at least 80 wt. % xylene isomers and a pX/X of less than 12 wt. %. The term “pX/X” refers to the weight percent of p-xylene (pX) in a referenced stream or product with respect to the total xylenes in the same stream or product (i.e., the sum of o-xylene, m-xylene, and p-xylene).

Suitable conditions for contacting the hydrocarbon-containing feed stream with the isomerization catalyst include liquid, vapor, or gaseous (supercritical) phase conditions in the presence or substantial absence of hydrogen. In certain embodiments, the hydrocarbon-containing feed stream is contacted with the isomerization catalyst in the presence of hydrogen. In certain other embodiments, the hydrocarbon-containing feed stream is contacted with the isomerization catalyst in the absence of hydrogen.

Typical vapor phase reaction conditions include a temperature of from about 500° F. to about 1000° F. In certain embodiments, the temperature is from about 600° F. to about 850° F. In certain embodiments, the temperature is from about 700° F. to about 800° F.

Typical vapor phase reaction pressure can be from about 0 psig to about 500 psig. In certain embodiments, the pressure can be from about 100 to about 300 psig.

Typical vapor phase reaction may also include an H₂/hydrocarbon mole ratio of from about 0 to 10. In certain embodiments, the H₂/hydrocarbon mole ratio is from about 0.5 to about 4.

Typical vapor phase reaction may also include a liquid weight hourly space velocity (LWHSV) of hydrocarbon-containing feed stream from about 1 to about 100. In certain embodiments, the LWHSV is from about 4 to about 15.

For example, in one embodiment the pressure is from about 0 psig to about 500 psig, the H₂/hydrocarbon mole ratio is from about 0 to about 10, and the liquid weight hourly space velocity (LWHSV) is from about 1 to about 100. In certain embodiments, vapor phase reaction conditions for xylene isomerization include a temperature of from about 600° F. to about 850° F., a pressure of from about 100 to about 300 psig, an H₂/hydrocarbon mole ratio of from about 0.5 to about 4, and a LWHSV of from about 4 to about 15. Other typical vapor phase conditions for xylene isomerization are further described, for example, in U.S. Pat. No. 4,327,236.

Typical liquid phase conditions for xylene isomerization are described, for example, in U.S. Pat. No. 4,962,258. The liquid phase process temperature can be from about 350° F. to about 650° F., or from about 500° F. to about 650° F.; or from about 550° F. to about 650° F. The upper temperature of the range is chosen so that the hydrocarbon feed to the process will remain in the liquid phase. The lower temperature limit can be dependent on the activity of the catalyst composition and may vary depending on the particular catalyst composition used. The total pressure used in the liquid phase process should be high enough to maintain the hydrocarbon feed to the reactor in the liquid phase, but there is no upper limit for the total pressure useful in the process. In certain embodiments, the total pressure is in the range of about 400 psig to about 800 psig. The process weight hourly space velocity (WHSV) is typically in the range of about 1 to about 60 hr⁻¹; or from about 1 to about 40 hr⁻¹; or from about 1 to about 12 hr⁻¹. Hydrogen may be used in the process, up to the level at which it is soluble in the feed; however, in certain embodiments, hydrogen is not used within the process. In another embodiment hydrogen is added above solubility but the bulk of the hydrocarbons remain in a liquid phase, for example in a trickle bed reactor.

Typical conditions for xylene isomerization at supercritical temperature and pressure conditions are described, for example, in U.S. Pat. No. 5,030,788. Generally, supercritical conditions contact the isomerization catalyst at a temperature and pressure above the critical temperature and pressure of the mixture of components in said stream. For a typical hydrocarbon-containing feed stream including xylene isomers, the critical pressure is above about 500 psig and the critical temperature is above about 650° F. Hydrogen may optionally be added to the reactor feed stream, as a small amount of hydrogen may reduce the rate of catalyst deactivation. If hydrogen is added, it can be added at a level below its solubility in the isomerization stream at reactor pressure and at temperatures present in a feed-effluent heat exchanger to avoid the formation of a vapor phase and its associated low heat transfer coefficient.

In any of the preceding embodiments, the MFI aluminosilicate molecular sieve can be prepared using a silicon source including a compound of the formula, Si(OR)₄, wherein R is C₁₋₁₀alkyl or aryl Si(OR₁)(OR₂)(OR₃)(OR₄), wherein R₁R₂R₃R₄ is each independently C₁₋₁₀alkyl or aryl. For example, the silicon source can be a tetra(C₁₋₁₀)alkylorthosilicate (e.g., tetra(C₁₋₆alkyl)orthosilicate) or a tetraarylorthosilicate. Suitable silicon sources include for example tetramethylorthosilicate, tetraethylorthosilicate, and tetraphenylorthosilicate. In certain embodiments, the silicon source includes tetraethylorthosilicate (Si(OEt)₄). In certain other embodiments, the silicon source includes tetraphenylorthosilicate (Si(OPh)₄).

The term “alkyl,” means a straight or branched chain saturated hydrocarbon containing from 1 to 10 carbon atoms, unless otherwise specified. Representative examples of alkyl include, for example, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “aryl,” means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom within the napthyl or azulenyl ring. The fused monocyclic cycloalkyl or monocyclic heterocyclyl portions of the bicyclic aryl may, but need not, be substituted with one or two oxo- and/or thia-groups. Representative examples of the bicyclic aryls include, for example, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, and benzo[d][1,3]dioxol-5-yl.

The MFI aluminosilicate molecular sieve can be prepared by combining an aluminum source, a template, and one of the preceding silicon sources at a suitable temperature to form a reaction mixture. Suitable temperatures include, for example between −20° C. and 200° C. In certain embodiments, the temperature is between 0° C. and 40° C. In certain embodiments, the temperature is between 0° C. and 10° C.

The template may be any familiar to one skilled in the art for preparing MFI aluminosilicate molecular sieves, including, for example, tetra(C₁₋₁₀alkyl)ammonium compounds, such as tetra(C₁₋₁₀alkyl)ammonium hydroxide (e.g., tetrapropylammonium hydroxide) or a tetra(C₁₋₁₀alkyl)ammonium halide (e.g., tetra(propyl)ammonium bromide). Similarly, the aluminum source may be any familiar to those skilled in the art for preparing MFI zeolites, including, for example, an aluminum C₁₋₁₀alkanoate or an aluminum C₁₋₁₀alkoxide such as aluminum isopropoxide.

Following formation of the reaction mixture, the mixture may be warmed to room temperature, e.g., between 20° C. and 40° C. Byproducts (e.g., volatile alcohols) may be optionally removed from the reaction mixture according to standard methods, such as under reduced pressure (with or without applied heat), to yield a concentrated reaction mixture. The reaction mixture or the concentrated reaction mixture may be heated to a third temperature between 100° C. and 200° C. (e.g., between 150° C. and 200° C.) for a period of time suitable to yield a product mixture including a solid, for example in an autoclave at autogenous pressure. When the byproducts (e.g., volatile alcohols) were not removed from the reaction mixture prior to heating to the third temperature, such byproducts may be removed from the reaction mixture according to standard methods, such as under reduced pressure (with or without applied heat), prior to isolation of the solid from the product mixture. The solid is isolated from the product mixture, for example, by filtration or centrifugation; and the resulting solid can be calcined to yield the isomerization catalyst. The calcining is typically at a temperature between 400° C. and 600° C. (e.g., between 480° C. and 600° C.; or between 500° C. and 600° C.; or between about 480° C. and about 540° C.).

The MFI aluminosilicate molecular sieves can have average crystallite sizes ranging from about 10 nm to 10 μm. In certain embodiments, the sieves can have average crystallite sizes ranging from about 10 nm to about 1 μm; or about 10 nm to about 500 nm; or about 50 nm to about 1 μm; or about 50 nm to about 500 nm, and may be used as isomerization catalysts in the methods of the invention in pure form or may further include a support. Suitable supports include, for example alumina (such as Sasol Dispersal® P3 alumina, PHF alumina), and silica, and mixtures thereof. The support may be provided in a quantity to yield an isomerization catalyst including 1-99 wt. % MFI aluminosilicate molecular sieve, such as 10-50 wt. % MFI aluminosilicate molecular sieve and the remainder support. In other embodiments, the isomerization catalyst includes 10-30 wt. % MFI aluminosilicate molecular sieve and the remainder support. In other embodiments, the isomerization catalyst comprises less than 90 wt. % support; or less than 80 wt. % support; or less than 70 wt. % support; or less than 60 wt. % support; or less than 50 wt. % support; or less than 40 wt. % support; or less than 30 wt. % support; or less than 20 wt. % support; or less than 10 wt. % support; or less than 5 wt. % support.

A hydrogenation catalyst component may be added to the MFI aluminosilicate molecular sieves, with the hydrogenation catalyst component being a metal or metal compound with the metals chosen from Groups VI-X of the periodic table. Suitable metals or compounds include, for example, metals or compounds of Pt, Pd, Ni, Mo, Ru, Rh, Re and combinations thereof. In certain embodiments, the hydrogenation catalyst component is Mo or a Mo compound. Other promoters or modifiers may be added such as Sn or S. For example, if Pt is used, it may be desirable to alloy with Sn, or to provide a low level of sulfiding.

Again, referring to FIG. 1a, the pX enriched stream (102) produced from the reaction zone (100) may be further processed in a separation zone (120′). The separation zone can include at least a pX recovery zone to recover at least a portion of a pX product (104), and, in certain embodiments, a fractionization zone to recover at least a portion of byproducts, each from the pX enriched stream. Typical byproducts include, for example, transmethylation by products benzene, toluene, trimethylbenzene, methyl(ethyl)benzene, and the like, which may be isolated from the pX enriched stream by standard methods such as fractional distillation. In certain embodiments, the pX enriched stream is processed to recover benzene byproduct and/or toluene byproduct.

Methods for isolating the pX product in the pX recovery zone (120) include, for example, (a) fractional crystallization, (b) liquid phase adsorption to chromatographically separate pX from the other C₈ aromatics; (c) chromatographic separation over zeolite MFI or ZSM-8, which has been reacted with an organic radical-substituted silane; (d) adsorptive separation of p-xylene and ethylbenzene through the use of MFI or ZSM-8 zeolites which have been reacted with certain silanes; (e) by heating a mixture of C₈ aromatic hydrocarbons to 50° F.-500° F. (10° C.-260° C.) followed by an adsorption/desorption step in the presence of a molecular sieve or synthetic crystalline aluminosilicate zeolite as the adsorbent (e.g., MFI) to recover a first mixture of p-xylene and ethylbenzene and a second mixture including meta-xylene, ortho-xylene, and any C₉ and higher aromatics; the resulting p-xylene and ethylbenzene mixture can be subjected to crystallization to recover p-xylene and the mother liquor can be subjected to distillation to recover the ethylbenzene; and (f) as disclosed in U.S. Pat. No. 6,573,418, by pressure swing adsorption employing a para-selective adsorbent (e.g., a large crystal, non-acidic medium pore molecular sieve) in connection with simulated moving bed adsorption chromatography.

The pX-lean stream (107) produced from the separation zone (120′) after generation of a pX product (e.g., a reject stream from a crystallization process or a raffinate from an adsorption process), containing relatively high proportions of EB, oX and mX, may be recycled to the reaction zone (100) for use as a hydrocarbon-containing feed stream (101′), or for combination with a hydrocarbon-containing feed stream (101).

As a result of the particular isomerization catalysts, the methods of the invention can provide a pX enriched stream (102) that contains reduced concentrations of byproducts of transmethylation as compared to similar methods using industry-standard xylene isomerization catalysts, such as AMSAC-3200. For example, the pX enriched stream can contain 3.5 wt. % or less net C₉-byproducts and/or 1.5 wt. % or less net toluene byproduct. The phrase “net byproduct,” refers to weight % of the referenced byproduct in an outgoing stream (e.g., “the pX enriched stream”) less the weight percent of the same “byproduct” in the incoming feed stream (e.g., “hydrocarbon-containing feed stream”). For example, where an incoming hydrocarbon-containing feed stream contains 1 wt. % of a byproduct (e.g., toluene) and the corresponding pX enriched stream contains 5 wt. % of the same byproduct, the pX enriched stream contains 4 wt. % net byproduct (e.g., 4 wt. % net toluene). The term “Cₙ-byproducts” refers to all chemical compounds in the referenced stream or product having “n” carbons in their individual chemical structures. For example, trimethylbenzene is a C₉-byproduct as it contains nine carbons in its chemical structure. In certain embodiments, the byproducts are aromatic compounds. Thus, in certain embodiments, the pX enriched stream can contain 3.5 wt. % or less net C₉-byproducts; or 3.0 wt. % or less; or 2.5 wt. % or less; or 2.0 wt. % or less net C₉-byproducts (e.g., C₉-aromatic byproducts). In other embodiments, the pX enriched stream can contain 1.5 wt. % or less net toluene byproduct; or 1.4 wt. % or less net toluene byproduct; or 1.3 wt. % or less net toluene byproduct; or 1.2 wt. % or less net toluene byproduct; or 1.1 wt. % or less net toluene byproduct; or 1.0 wt. % or less net toluene byproduct; or 0.9 wt. % or less net toluene byproduct; or 0.8 wt. % or less net toluene byproduct.

In other embodiments, the pX enriched stream contains less than 0.7 wt. % net trimethylbenzene byproduct; or less than 0.6 wt. % net trimethylbenzene byproduct or; less than 0.5 wt. % net trimethylbenzene byproduct.

The present methods provide a pX enriched stream containing at least 23.5 wt. % pX/X. In one embodiment, the pX enriched stream contains at least 23.5 wt. % pX/X and less than 1.5 wt. % net toluene byproduct. In another embodiment, the pX enriched stream contains at least 23.5 wt. % pX/X and less than 1.0 wt. % net toluene byproduct. In another embodiment, the pX enriched stream contains at least 23.8 wt. % pX/X and less than 1.5 wt. % net toluene byproduct. In another embodiment, the pX enriched stream contains at least 23.8 wt. % pX/X and less than 1.0 wt. % net toluene byproduct.

In yet other embodiments, the present methods provide a pX enriched stream containing at least 23.8 wt. % pX/X and less than 0.6 wt. % net trimethylbenzene byproduct. In yet other embodiments, the present methods provide a pX enriched stream containing at least 23.8 wt. % pX/X and less than 0.5 wt. % net trimethylbenzene byproduct.

In further embodiments, the present methods provide a pX enriched stream containing at least 23.5 wt. % pX/X and a ratio of pX/X to the sum of net wt. % trimethylbenzene byproduct and net wt. % toluene byproduct of greater than 4.0 (e.g., between 4.0 and 10.0). In other embodiments, the pX enriched stream contains at least 23.6 wt. % pX/X; or at least 23.7 wt. % pX/X; or at least 23.8 wt. % pX/X and a ratio of pX/X to the sum of net wt. % trimethylbenzene byproduct and net wt. % toluene byproduct of greater than 4.0 (e.g., between 4.0 and 10.0, or between 4.0 and 8.0).

In other embodiments, the pX enriched stream contains at least 23.5 wt. % pX/X; or at least 23.6 wt. % pX/X; or at least 23.7 wt. % pX/X; or at least 23.8 wt. % pX/X and a ratio of pX/X to the sum of net wt. % trimethylbenzene byproduct and net wt. % toluene byproduct of greater than 5.0 (e.g., between 5.0 and 10.0, or between 5.0 and 8.0).

In other embodiments, the pX enriched stream contains at least 23.5 wt. % pX/X; or at least 23.6 wt. % pX/X; or at least 23.7 wt. % pX/X; or at least 23.8 wt. % pX/X and a ratio of pX/X to the sum of net wt. % trimethylbenzene byproduct and net wt. % toluene byproduct of greater than 6.0 (e.g., between 6.0 and 10.0, or between 6.0 and 8.0).

In other embodiments, the pX enriched stream contains at least 23.5 wt. % pX/X; at least 23.6 wt. % pX/X; at least 23.7 wt. % pX/X; or at least 23.8 wt. % pX/X; or essentially equilibrium pX concentration for the temperature of the reaction (e.g., 24.1 wt. % at between 700° F. and 750° F.).

In certain embodiments, as shown in FIG. 1b, the pX enriched stream (102) produced from the reaction zone can be further processed in a fractionization zone (110) to recover at least a portion of the byproducts (103) from the pX enriched stream. Typical byproducts and methods for isolation can be as described above. In certain embodiments, the pX enriched stream (102) is processed in the fractionization zone (110) to recover benzene byproduct and/or toluene byproduct. After removal of byproducts, at least a portion of the pX product (104) can be recovered in a pX recovery zone (120) from the pX enriched stream (102). The pX-lean stream (107) produced after generation of a pX product may be recycled to the reaction zone (100) for use as a hydrocarbon-containing feed stream (101′), or for combination with a hydrocarbon-containing feed stream (101).

Referring to FIG. 1c, in another embodiment, prior to recovery of the pX product (104), the pX enriched stream (102) may be combined with a make-up feed stream (105). The make-up feed stream (105) may be introduced, as shown by branch (105a), at the fractionation zone (110) to provide a combination stream (106) from the fractionation zone. The make-up feed stream (105a) provided to the fractionation zone (110) can be, for example, a C8+ reformate distillation cut of a refinery reformer. In this case, the fractionation zone (110) can remove byproducts (103) produced in reaction zone (100) and C9+ aromatics or other non-C8 aromatics that may be present in make-up feed stream (105). Alternatively, depending on the source of the make-up feed stream (e.g., where byproduct removal is not necessary), the make-up feed stream (105) may be introduced, as shown by branch (105b), after the fractionation zone (110) to provide the combination stream (106). Then, at least a portion of the pX product (104) may be recovered from the combination stream (106) in a recovery zone (120). The resulting pX-lean stream (107) can be recycled in any of the preceding methods to the reaction zone (100) for use as the hydrocarbon-containing feed stream (101′), or for combination with a hydrocarbon-containing feed stream (101).

Thus, in one embodiment, as shown in FIG. 1c, a reaction zone (100) comprises a reactor with a catalyst or dual bed catalyst system comprising a boroaluminosilicate molecular sieve prepared according to this invention. The reaction zone (100) isomerizes the xylenes and converts some of the ethylbenzene in the hydrocarbon-containing feed stream (101 or 101′) producing a pX enriched stream (102), while producing some byproducts including benzene, toluene and A9+ aromatics. At least a portion of the byproducts produced are separated in fractionation zone (110) to produce byproducts stream(s) (103). The pX enriched stream freed of some byproducts is combined with a make-up feed stream (105b) comprising the xylene isomers and ethylbenzene to produce a combination stream (106) which is fed to a pX recovery zone (120). Alternatively, a make-up stream (105a), for example, a C8+ reformate distillation cut of a refinery reformer, is fed to the fractionation zone (110), and the combination stream (106) produced from the fractionation zone. Then, at least a portion of the pX in the combination stream (106) is removed in a pX recovery zone (120) as a pX product stream (104). The pX recovery zone (120) also produces a pX lean stream (107) which is recycled to reaction zone (100) as the hydrocarbon-containing stream (101) or for combination with a hydrocarbon-containing stream (101′).

The preceding methods may be practiced in conjunction with a dual-bed catalyst configuration. Accordingly, the methods may further include contacting the hydrocarbon-containing feed stream with an ethylbenzene (EB) conversion catalyst under conditions suitable to reduce the EB content of the hydrocarbon-containing feed stream. Such contacting may occur, for example, prior to contacting the hydrocarbon-containing feed stream with the isomerization catalyst. In certain embodiments, the hydrocarbon-containing feed stream is contacted with the EB conversion catalyst and the isomerization catalyst in a single reaction zone.

Suitable ethylbenzene conversion catalysts include, for example, AI-MFI aluminosilicate molecular sieve dispersed on silica and large particle size molecular sieves, such as MFI aluminosilicate molecular sieve having a particle size of at least about 1 μm, dispersed on silica, alumina, silica/alumina or other suitable support. In one example, the EB conversion catalyst includes an Al-MFI aluminosilicate molecular sieve having a particle size of at least about 1 μm supported on Cab-o-sil® HS-5 (a high surface fumed silica available from Cabot Corporation, Billerica, Mass.) with a compound of Mo added. Suitable catalysts based on a ZSM-type molecular sieve, for example, MFI aluminosilicate molecular sieves. In addition, other types of molecular sieve catalysts can also be used (e.g., ZSM-11, ZSM-12, ZSM-35, ZSM-38 and other similar materials).

A hydrogenation catalyst component may be added to the ethylbenzene conversion catalyst, with the hydrogenation catalyst component being a metal or metal compound with the metals chosen from Groups VI-X of the periodic table, as noted above for the isomerization catalysts. In certain embodiments, the hydrogenation catalyst component is Mo or a Mo compound. Other promoters or modifiers may be added such as Sn or S. For example, if Pt is used, it may be desirable to alloy with Sn, or to provide a low level of sulfiding. In other embodiments, both the isomerization catalyst and the ethylbenzene conversion catalyst comprise a hydrogenation catalyst component. In certain embodiments, both catalysts comprise Mo or a Mo compound.

The ethylbenzene conversion catalyst may include about 1% to about 100% by weight of molecular sieve, or about 10 to about 70% by weight, with the remainder being support matrix material such as alumina or silica, or a mixture thereof. In certain embodiments, the support material is silica. In certain embodiments, the support material is alumina. In certain embodiments the support is a combination of silica and alumina. The weight ratio of ethylbenzene conversion catalyst to isomerization catalyst can be about 0.25:1 to about 6:1.

Catalyst Systems

In another aspect, the present invention provides catalyst systems for use in any of the preceding methods and embodiments of the same. In particular, the catalyst systems are useful in methods for enriching a xylene isomers feed in p-xylene. Such catalyst systems include dual bed configurations including a first bed including an ethylbenzene (EB) conversion catalyst and a second bed including an isomerization catalyst that is a MFI aluminosilicate molecular sieve prepared using a silicon source including a compound of the formula, Si(OR₁)(OR₂)(OR₃)(OR₄), wherein R₁R₂R₃R₄ is each independently C₁₋₁₀alkyl or aryl.

For example, the MFI aluminosilicate molecular sieve of the catalyst system is prepared using a silicon source including a compound of the formula, Si(OR₁)(OR₂)(OR₃)(OR₄), wherein R₁R₂R₃R₄ is each independently C₁₋₁₀alkyl or aryl. For example, the silicon source can be a tetra(C₁₋₁₀)alkylorthosilicate (e.g., tetra(C₁₋₆alkyl)orthosilicate) or a tetraarylorthosilicate. Suitable silicon sources include for example tetramethylorthosilicate, tetraethylorthosilicate, and tetraphenylorthosilicate. In certain embodiments, the silicon source includes tetraethylorthosilicate (Si(OEt)₄). In certain other embodiments, the silicon source includes tetraphenylorthosilicate (Si(OPh)₄).

The MFI aluminosilicate molecular sieve can be prepared by combining an aluminum source, a template, and one of the preceding silicon sources at a suitable temperature to form a reaction mixture. Suitable temperatures include, for example between −20° C. and 200° C. In certain embodiments, the temperature is between 0° C. and 40° C. In certain embodiments, the temperature is between 0° C. and 10° C.

The template may be any familiar to one skilled in the art for preparing MFI aluminosilicate molecular sieves, including, for example, tetra(alkyl)ammonium compounds, such as tetra(alkyl)ammonium hydroxide (e.g., tetrapropylammonium hydroxide) or a tetra(alkyl)ammonium halide (e.g., tetra(propyl)ammonium bromide). Similarly, the aluminum source may be any familiar to those skilled in the art for preparing MFI zeolites, including, for example, aluminum alkanoate such as aluminum isopropoxide.

Following formation of the reaction mixture, the mixture may be warmed to room temperature, e.g., between 20° C. and 40° C. Byproducts (e.g., volatile alcohols) may be optionally removed from the reaction mixture according to standard methods, such as under reduced pressure (with or without applied heat), to yield a concentrated reaction mixture. The reaction mixture or the concentrated reaction mixture may be heated to a third temperature between 100° C. and 200° C. (e.g., between 150° C. and 200° C.) for a period of time suitable to yield a product mixture including a solid, for example in an autoclave at autogenous pressure. When the byproducts (e.g., volatile alcohols) were not removed from the reaction mixture prior to heating to the third temperature, such byproducts may be removed from the reaction mixture according to standard methods, such as under reduced pressure (with or without applied heat), prior to isolation of the solid from the product mixture. The solid is isolated from the product mixture, for example, by filtration or centrifugation; and the resulting solid can be calcined to yield the isomerization catalyst. The calcining is typically at a temperature between 400° C. and 600° C. (e.g., between 480° C. and 600° C.; or between 500° C. and 600° C.; or between about 480° C. and about 540° C.).

The MFI aluminosilicate molecular sieves can have average crystallite sizes ranging from about 10 nm to 10 μm. In certain embodiments, the sieves can have average crystallite sizes ranging from about 10 nm to about 1 μm; or about 10 nm to about 500 nm; or about 50 nm to about 1 μm; or about 50 nm to about 500 nm, and may be used as isomerization catalysts in the methods of the invention in pure form or may further include a support. Suitable supports include, for example alumina, such as Sasol Dispersal® P3 alumina, PHF alumina, and silica, and mixtures thereof. The support may be provided in a quantity to yield an isomerization catalyst including 1-99 wt. % MFI aluminosilicate molecular sieve, such as 10-50 wt. % MFI aluminosilicate molecular sieve and the remainder support. In other embodiments, the isomerization catalyst includes 10-30 wt. % MFI aluminosilicate molecular sieve and the remainder support. In other embodiments, the isomerization catalyst comprises less than 90 wt. % support; or less than 80 wt. % support; or less than 70 wt. % support; or less than 60 wt. % support; or less than 50 wt. % support; or less than 40 wt. % support; or less than 30 wt. % support; or less than 20 wt. % support; or less than 10 wt. % support; or less than 5 wt. % support.

A hydrogenation catalyst component may be added to the MFI aluminosilicate molecular sieves, with the hydrogenation catalyst component being a metal or metal compound with the metals chosen from Groups VI-X of the periodic table. Suitable metals or compounds include, for example, metals or compounds of Pt, Pd, Ni, Mo, Ru, Rh, Re and combinations thereof. In certain embodiments, the hydrogenation catalyst component is Mo or a Mo compound. Other promoters or modifiers may be added such as Sn or S. For example, if Pt is used, it may be desirable to alloy with Sn, or to provide a low level of sulfiding.

Suitable ethylbenzene conversion catalysts include, for example, AI-MFI aluminosilicate molecular sieve dispersed on silica and large particle size molecular sieves, such as MFI aluminosilicate molecular sieve having a particle size of at least about 1 μm, dispersed on silica, alumina, silica/alumina or other suitable support. In one example, the EB conversion catalyst includes MFI aluminosilicate molecular sieve having a particle size of at least about 1 μm supported on Cab-o-sil® HS-5 (a high surface fumed silica available from Cabot Corporation, Billerica, Mass.) with a compound of Mo added. Suitable catalysts based on a MFI aluminosilicate molecular sieve. In addition, other types of molecular sieve catalysts can also be used (e.g., ZSM-11, ZSM-12, ZSM-35, ZSM-38 and other similar materials).

As noted, a hydrogenation catalyst component may be added to the ethylbenzene conversion catalyst, with the hydrogenation catalyst component being a metal or metal compound with the metals chosen from Groups VI-X of the periodic table, as noted above for the isomerization catalysts. In certain embodiments, the hydrogenation catalyst component is Mo or a Mo compound. Other promoters or modifiers may be added such as Sn or S. For example, if Pt is used, it may be desirable to alloy with Sn, or to provide a low level of sulfiding. In other embodiments, both the isomerization catalyst and the ethylbenzene conversion catalyst comprise a hydrogenation catalyst component. In certain embodiments, both catalysts comprise Mo or a Mo compound.

The ethylbenzene conversion catalyst may include about 1% to about 100% by weight of molecular sieve, or about 10 to about 70% by weight, with the remainder being support matrix material such as alumina or silica, or a mixture thereof. In certain embodiments, the support material is silica. In certain embodiments, the support material is alumina. The weight ratio of ethylbenzene conversion catalyst to isomerization catalyst is suitably about 0.25:1 to about 6:1.

In certain embodiments, the first bed, including the EB conversion catalyst is disposed over the second bed, including the MFI aluminosilicate molecular sieve.

The phrase “disposed over” means that the first referenced item (e.g., first bed) can be in direct contact with the surface of the second referenced item (e.g., second bed), or one or more intervening materials or structures may also be present between the surface of the first item (e.g., first bed) and the surface of the second item (e.g., second bed). However, when one or more intervening materials or structures are present (such as screens to support and/or separate the first and second beds), the first and second items, nonetheless, remain in fluid communication with each other (e.g., the screens allow for the hydrocarbon-containing feed stream to pass from the first bed to the second bed). Further, the first item (e.g., first bed) may cover the entire surface or a portion of the surface of the second item (e.g., second bed). Alternatively, the catalyst system includes a guard bed, including a hydrogenation catalyst component, disposed over the first bed. A guard bed may also be disposed between the first bed and the second bed. The weight ratio of ethylbenzene catalyst to hydrogenation catalyst component can be about 1:1 to about 20:1.

The hydrogenation catalyst component may contain a hydrogenation metal, such as molybdenum, platinum, palladium, rhodium, ruthenium, nickel, iron, osmium, iridium, tungsten, rhenium, and the like, and may be dispersed on a suitable matrix. Suitable matrix materials include, for example, alumina and silica. Although a molybdenum-on-alumina catalyst is effective, other hydrogenation catalyst components, for example those including platinum, palladium, rhodium, ruthenium, nickel, iron, osmium, iridium, tungsten, rhenium etc., deposited on a suitable support such as alumina or silica may also be used. It is advantageous to avoid hydrogenation catalyst components and/or reaction conditions that cause aromatic ring hydrogenation of the xylenes. When molybdenum-on-alumina is used, the level of molybdenum can be about 0.5 to about 10 weight percent, or about 1 to about 5 weight percent.

In another aspect, the invention provides xylene isomerization reactor including a reaction zone containing the catalyst system as described above. The xylene isomerization reactor can be a fixed bed flow, fluid bed, or membrane reactor containing the catalyst system described above. The reactor can be configured to allow a hydrocarbon-containing feed stream to be cascaded over the catalyst system disposed in a reaction zone in sequential beds; for example, first, the EB conversion catalyst bed and then the xylene isomerization catalyst bed; or first, the xylene isomerization catalyst and then the EB conversion catalyst In another embodiment, first, the EB conversion catalyst bed, then, a “sandwiched” hydrogenation catalyst component bed, and finally, the xylene isomerization catalyst bed. Alternatively, first, the xylene isomerization catalyst bed, then, the “sandwiched” hydrogenation catalyst component bed, and finally, the EB conversion catalyst bed. In another embodiment, the reactor may include separate sequential reactors wherein the feed stream would first be contacted with the EB conversion catalyst in a first reactor, the effluent from there would be optionally contacted with the “sandwiched” hydrogenation catalyst component in an optional second reactor, and the resulting effluent stream would then be contacted with the xylene isomerization catalyst in a third reactor. In another embodiment, the xylene isomerization catalyst bed may comprise a hydrogenation catalyst component disposed over the EB conversion catalyst and another “sandwiched” hydrogenation catalyst component between the EB conversion catalyst and the isomerization catalyst.

While specific embodiments have been described in detail, and in particular in the following Examples, those with ordinary skill in the art will appreciate that various modifications and alternatives could be developed in light of the overall teachings of the disclosure. Accordingly, the particular arrangements disclosed are meant to be illustrative only and not limiting as to the scope of the invention, which is to be given the full breadth of the appended claims, including any and all equivalents thereof. Unless otherwise defined, all technical and scientific terms used herein have the same meaning as commonly understood by one of ordinary skill in the art to which this invention pertains. All references mentioned in this description, including publications, patent applications, and patents, are incorporated by reference in their entirety. In addition, the materials, methods, and examples described are only illustrative and not intended to be limiting.

### EXAMPLES

### Example 1

### Preparation of MFI Aluminosilicate Molecular Sieves

(a) General Preparation

Precursors such as silica sol, an aluminum compound, tetrapropylammonium template, and base were mixed and charged into 125-cc Parr reactors. These reactors were sealed and then heated at 150-170° C. for 2-5 days in an oven. Agitation of the reactor contents was accomplished by rotational tumbling of the reactors inside the temperature-controlled oven. The oven could accommodate up to 12 reactors simultaneously. Product work-ups involved standard filtration, water-washing, and drying methods. Final products were typically calcined at 538° C. (1000° F.) for 5 hours.

(b) “Conventional” MFI Aluminosilicates

“Conventional” MFI aluminosilicates were made using an aqueous mixture of the silica sol, aluminum sulfate or sodium aluminate, template (tetrapropylammonium bromide), and base (NaOH), followed by ammonium acetate exchange to remove sodium.

(c) MFI Aluminosilicate Molecular Sieves from TEOS

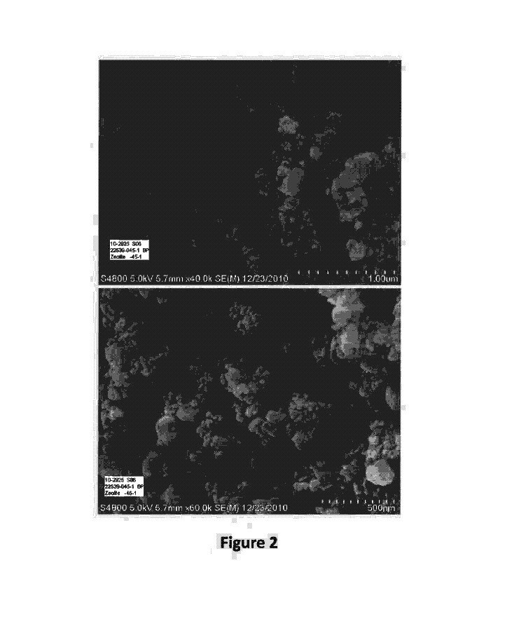

MFI aluminosilicate molecular sieves using tetraethylorthosilicate (TEOS, Si(OEt₄) as the Si source were prepared following the general method of Van Grieken et al., Microporous and Mesoporous Materials 39 (2000) 135-147. Aluminum isopropoxide (5.76 g) was added to 300 g TPAOH (tetrapropylammonium hydroxide, 40 wt. % aqueous solution, TCI America) in a 1-liter flask at room temperature. The mixture was cooled to 4° C. with an ice bath and stirred to obtain a clear solution. TEOS (tetraethyl orthosilicate, 99+%, Sigma Aldrich, 176.4 g) was added dropwise to the cooled aluminum isopropoxide/TPAOH solution over about an hour using an addition funnel. The solution was maintained at 4° C. for most of this time, although the temperature warmed to 16° C. as the last of the TEOS was added. The vessel was removed from the ice bath and stirred at room temperature for 40 hours. Alcohol products (˜182 g, mainly ethanol produced from TEOS hydrolysis) were distilled off using a rotary evaporator at 79° C. under vacuum (22″ Hg) over 2.5 hours. Approximately 291 g (250 mL, ˜1.16 g/cc) of concentrated solution remained after evaporative removal of alcohols, and that concentrate was used later in the Parr reactors for MFI synthesis as described above (heating at 170° C. for 2-5 days). An example SEM image of a MFI prepared from TEOS is shown in FIG. 2. This sample contains 1.5 wt. % Al, is 99% crystalline, and is comprised of very small sub-micron crystals.

### Example 2

### Comparative Catalytic Activity Study

Samples of “commercial” zeolite molecular sieves and catalysts were obtained from Tosoh, Zeolyst, TriCat, Qingdao Wish Chemical, and Zibo Xinhong Chemical Trade Co. (see Table 1). The TriCat and Tosoh “HSZ-820NAA” samples were ammonium-exchanged by a conventional procedure: an ammonium acetate solution was made by dissolving 1 g ammonium acetate in 10 g deionized (DI) water (such as 100 g ammonium acetate in 1000 g DI water). Then 1 g of the sieve to be exchanged was added to 11 g of the ammonium acetate solution. The mixture was heated to 85° C. for one hour while stirring, filtered using a vacuum filter, and washed with 3 aliquots of 3 g DI water per g of sieve while the sieve was still on the filter paper. The sieve was re-slurried in 11 g of fresh ammonium acetate solution, heated to 85° C. on a heating pad for one hour while stirring, filtered and washed with DI water as per above. It was then dried and calcined in air: 4 hrs at 329° F., ramp to 900° F. over 4 hours, calcined for 4 h. at 900° F.

Commercial MFI aluminosilicate catalysts were tested unsupported (i.e., as “pure” sieves) and were supported on alumina (20% sieve, 80% alumina) according to the following procedure:

40 g Sasol Disperal® P3 alumina (Sasol Germany GmbH, Hamburg, Germany) was added to 360 g of 0.6 wt. % deionized distilled (DD) water to form an alumina sol, and homogenized for 15 minutes. A mixture of 8 g of sieve in 24 g DD water was prepared and homogenized for 3 minutes. 320 g of the alumina sol was placed into a beaker and the sieve/DD water mixture was added, followed by homogenization for 5 minutes. After standing for 30 minutes, the sieve/sol mixture was transferred to a kitchen blender and 24 mL of concentrated ammonium hydroxide (nominal 28 wt. % ammonia) was added. The resulting gel was mixed at setting 4 for 5 minutes. The mixture was poured into a drying dish (about 2 inch depth), dried for 4 h. at 329° F., ramped to 900° F. over 4 hours, and finally calcined at 900° F. for 4 hours.

The Following Catalysts were Prepared as Controls:
1. “AMSAC-3200 P3” containing nominal 20 wt. % HAMS-1B-3 borosilicate molecular sieve (hydrogen form of AMS-1B) and 80 wt. % Sasol Disperal® P3 alumina
2. “AMSAC-3200”, commercial, nominal 20 wt. % borosilicate molecular sieve with 80 wt. % alumina binder.
3. “AMSAC-3202M”, commercial, nominal 20 wt. % borosilicate molecular sieve with 80 wt. % alumina binder, contains 2 wt. % Mo.

Catalytic Testing

The catalysts were charged into 2-mm ID tube reactors as powders (50-200 μm) in a high-throughput catalyst testing apparatus consisting of 16 parallel fixed-bed flow reactors. The catalysts were activated by heating the reactors under H₂ flow without hydrocarbon feed for at least an hour at reaction temperature prior to introducing hydrocarbon feed. Then, hydrogen gas and the xylene isomers were combined and fed to the reactor. Reactor effluent hydrocarbons were analyzed every 4 hours by an on-line gas chromatograph.

The feed stream of xylene isomers contained 1.03 wt. % benzene, 1.98 wt. % toluene, 10.57 wt. % EB (ethylbenzene), 9.75 wt. % pX (p-xylene), 50.22 wt. % mX (m-xylene), and 24.16 wt. % oX (o-xylene), corresponding to 11.6% pX isomer in the xylene isomers.

A first testing phase was conducted to screen and rank catalysts for xylene isomerization activity. Relatively mild conditions were employed (600° F., 38 h⁻¹ WHSV xylenes feed, 225 psig, 1.5 H₂/hydrocarbon mole ratio and LWHSV=38 based on 20 wt % sieve catalysts with LWHSV adjusted based on sieve content when testing unsupported sieves) to discriminate based on activity for xylene isomerization. EB conversions were very low, <10%, under these mild conditions. Isomerization of xylenes to theoretical equilibrium would yield about 24.1% pX/xylenes in the reactor effluent. Reactor effluents were sampled periodically during the runs and analyzed by gas chromatography. Catalysts were observed to undergo moderate deactivation over 50+ hours on stream. Due to the deactivation, % pX/xylenes results were calculated as averages over the first 40-50 hours on stream.

Each run (block of 16 reactors) included at least two of the AMSAC-3200 and/or AMSAC-3202M reference catalysts as controls. The performance of the AMSAC references was reproducible from run to run

Of the 60 catalysts tested, 17 were found to isomerize xylenes with similar effectiveness as the AMSACs (20-23% pX/xylenes), including 12 commercial MFI materials and the MFI catalysts prepared from TEOS. The remaining catalysts were less active, with about a dozen being essentially inactive, Table 1 presents a summary of the most active catalysts in the first phase of testing, where “S” indicates the sieve was tested in pure form and “C” indicates that the sieve was supported on alumina, as prepared above.

| | | % pX/ | Sieve (S) | Al |
|---|---|---|---|---|
| | | Xylenes | or Alumina- | wt. % |
| | | (avg of | supported | in |
| Catalyst | Type | 2 trials) | (C) | sieve |
| Control Catalysts | | | | |
| AMSAC-3200 | borosilicate | 21% | C | |
| AMSAC-3200 P3 | borosilicate | 22% | C | |
| AMSAC-3202M | borosilicate | 21% | C | |
| Commercial Catalysts | | | | |
| Tosoh (Grove City, OH) | | | | |
| HSZ ®-820NAA, H-form | MFI | 21% | S | 3.4 |
| HSZ ®-820NAA, H-form | MFI | 23% | C | 3.4 |
| Zeolyst Int'l. | | | | |
| (Conshohocken, PA) | | | | |
| CBV 2314 | MFI | 20% | S | 3.4 |
| CBV 2314CY1.6 | MFI | 20% | S | 3.4 |
| CBV 3024E | MFI | 21% | S | 2.5 |
| CBV 3024E | MFI | 22% | C | 2.5 |
| CBV 3014CY1.6 | MFI | 21% | C | 2.5 |
| CBV 5524G | MFI | 20% | S | 1.5 |
| TriCat Catalysts | | | | |
| (Hunt Valley, MD) | | | | |
| TriCat | MFI | 21% | S | 3.3 |
| TriCat | MFI | 23% | C | 3.3 |
| Zibo Xinhong Chemical | | | | |
| (Zibo City, China) | | | | |
| Zibo Xinhong Chemical | MFI | 21% | S | 2.5 |
| Zibo Xinhong Chemical | MFI | 22% | C | 2.5 |
| Example 1(b) | MFI | 23% | S | 2.2 |
| Example 1(b) | MFI | 19% | S | 2.2 |
| Example 1(b) | MFI | 19% | S | 2.2 |
| Example 1(c) | MFI (TEOS) | 21% | S | 1.5 |
| Example 1(c) | MFI (TEOS) | 19% | S | 1.5 |
| Example 1(c) | MFI (TEOS) | 19% | S | 1.5 |

For the MFI catalysts, there was a general trend of increasing isomerization activity with higher Al content in the zeolite molecular sieve. This is often true for reactions catalyzed by acidic zeolites such as MFI aluminosilicate molecular sieves. There was also a trend toward higher EB conversion with higher Al content.

### Example 3

### Commercial Conditions Testing

Based on the results of Example 2, approximately thirty isomerization catalysts were tested at higher temperatures (650-770° F.) that are more typical of a commercial PX reactor, to determine isomerization activity and selectivity at higher EB conversions (20-70%). For selectivity, the extent of xylene loss reactions through transmethylation processes was measured, such as the methyl transfer reactions.

Data was collected at five different temperatures (650° F., 680° F., 710° F., 740° F., 770° F.) at 10 h⁻¹ WHSV xylenes feed, 225 psig, and 1.5 H₂/hydrocarbon mole ratio. Typically, three reactor effluent samples were taken at each temperature and analyzed by gas chromatography. Averages of the three sample analyses were calculated.

Ethylbenzene conversions were observed at each of the five tested temperatures. In general, it was observed that the commercial and conventionally-made MFI sieves showed the highest activity for EB conversion, the AMSAC references exhibited the lowest activity, and the TEOS-prepared MFI sieves were intermediate. In contrast, activities for xylene isomerization were nearly the opposite. The commercial and conventionally-made MFI sieves displayed significantly lower isomerization activities than most of the other catalysts. The best catalysts (AMSACs and TEOS-made MFI sieves) isomerized the xylenes to about 23.9-24.0% pX, near thermodynamic equilibrium (24.1% pX).

Viewed in terms of EB conversion versus xylene isomerization activity, the commercial and conventionally-prepared MFI aluminosilicate catalysts were largely inferior to the other catalyst groups, including the MFI aluminosilicate molecular sieves prepared from TEOS, in xylene isomerization activity over a wide range of EB conversions.

Catalyst selectivity was examined by comparing the relative amounts of undesirable products generated through transmethlation reactions. Toluene is produced through two transmethylation reactions: xylene disproportionation and methyl transfer from xylene (XYL) to EB. Other transmethylation products include trimethylbenzenes (TMB) and methylethylbenzenes (MEB). For catalysts containing hydrogenation catalyst components, toluene (TOL) can also be formed from secondary dealkylation of MEB:

$$XYL + EB = MEB + TOL$$

$$\underline{MEB + {H}_{2} \to TOL + {C}_{2}}$$

$$XYL + EB + {H}_{2} \to 2 TOL + {C}_{2} ( Net Reaction )$$

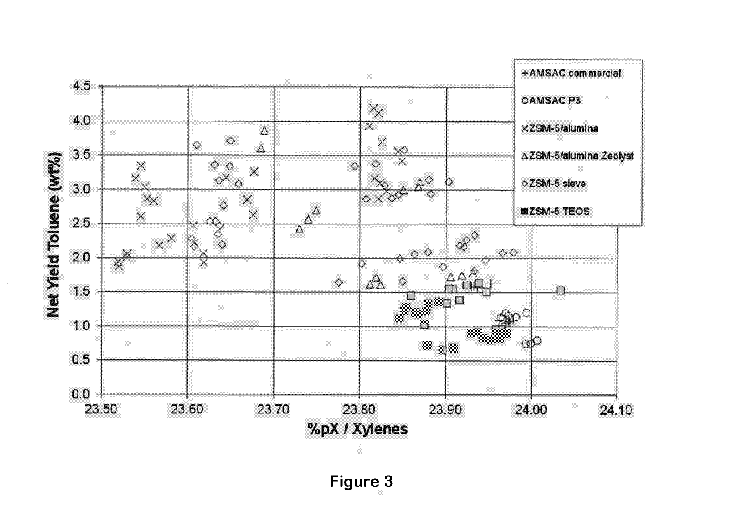

The amount of toluene in the reactor effluent (GC area %) was examined over a range of EB conversions for the catalyst groups. The AMSACs and TEOS-prepared MFI aluminosilicate catalysts yielded very similar and low amounts of toluene, whereas the commercial and conventionally-prepared MFI aluminosilicate catalysts yielded substantially more toluene. FIG. 3 is a graph of net toluene yield (toluene in feed has been subtracted out) as a function of xylene isomerization activity. Again, the AMSACs and TEOS-prepared MFI aluminosilicate catalysts yielded lower amounts of toluene relative to the other MFI aluminosilicate catalysts.

With respect to other byproducts, trimethylbenzenes and methylethylbenzenes, most of the commercial and conventionally-prepared MFI aluminosilicate catalysts yielded higher amounts of these than did the AMSACs and TEOS-prepared MFI aluminosilicate catalysts.

In summary, at the higher temperature conditions, MFI sieves prepared from TEOS exhibited high xylene isomerization activity (23.9-24.0% pX/xylenes) that was very similar to the performance of AMSAC-3200 reference catalysts in first testing stage. The catalysts also produced low xylene losses from transmethylation reactions (to toluene, trimethylbenzenes, and methylethylbenzenes) over a wide range of EB conversions (20-70%), also similar to the performance of AMSAC-3200 reference catalysts.

However, in contrast, commercial and conventionally-prepared MFI catalysts performed poorly and showed relatively low isomerization activity under these conditions (less than 23.9% PX/xylenes) and higher activity for undesirable xylene transmethylation (xylene loss) reactions. Notably, the TEOS-made MFI aluminosilicate catalysts do not require alumina activation, and in fact, were tested only in pure sieve form.

### Example 4

### Quantification of Byproducts

MFI zeolites were prepared with TEOS as a silicon source as described above; Al contents were determined to be 1.4-1.5 wt. % by ICP. SEM indicated that the average crystallite sizes were below 1 μm in size, ranging from about 50 nm to about 500 nm. MFI catalysts were tested for isomerization of xylenes using small fixed-bed flow reactors with a commercial “xylene isomers” aromatics feed consisting of 1.03 wt % benzene, 1.98% toluene, 10.57% ethylbenzene, 9.75% p-xylene, 50.22% m-xylene, and 24.16% o-xylene (11.6% p-xylene in total xylenes).

The catalysts were charged into 2-mm ID tube reactors as powders (50-200 μm). Hydrogen gas and the xylene isomers were combined and fed to the reactor in a 1.5 mole ratio (H₂/hydrocarbon) at 225 psig and with a xylene isomers feed rate of 10 LWHSV (gm feed/gm catalyst-hr). Reactor temperature was either 650 or 680° F. Reactor effluent hydrocarbons were analyzed every 4 hours by an on-line gas chromatograph. A summary of the catalytic test results is given in Table 2.

TABLE 2
Comparison of MFI Catalysts for Xylene Isomerization

| | | | | | | | Sum of |
|---|---|---|---|---|---|---|---|
| | | Ethyl- | % p-Xylene | | Trimethyl- | Methyl- | Toluene, |
| | | benzene | in Reactor | Toluene | benzene | ethylbenzene | TMB, and |
| Catalyst | Temp | Conversion | Effluent | Yield | (TMB) Yield | (MEB) Yield | MEB Yields |
| (ZSM-5 zeolite in H⁺ form) | (° F.) | (%) | Xylene | (net, wt %) | (net, wt %) | (net, wt %) | (wt %) |
| Commercial ZSM-5's | | | | | | | |
| Tricat (20% zeolite on Al₂O₃) | 650 | 32.2 | 23.61 | 2.17 | 0.46 | 0.71 | 3.34 |
| Tosoh HSZ-820 (pure zeolite) | 650 | 38.3 | 23.92 | 2.24 | 0.94 | 0.69 | 3.87 |
| Zeolyst CBV 3014 (80% zeolite) | 650 | 32.7 | 23.92 | 1.75 | 0.83 | 0.63 | 3.21 |
| Zeolyst CBV 2314 (80% zeolite) | 650 | 36.6 | 23.74 | 2.56 | 0.79 | 0.85 | 4.20 |
| Zeolyst CBV 5524G (80% zeolite) | 680 | 37.6 | 23.82 | 1.64 | 0.79 | 0.76 | 3.19 |
| Average of the five commercial ZSM-5's | | 35.5 | 23.80 | 2.07 | 0.76 | 0.73 | 3.56 |
| ZSM-5's prepared from TEOS* | | | | | | | |
| ZSM-5 #1 pure zeolite (3 days crystallization) | 680 | 34.9 | 23.94 | 0.92 | 0.51 | 0.44 | 1.87 |
| ZSM-5 #2 pure zeolite (2 days crystallization) | 680 | 32.9 | 23.90 | 0.66 | 0.49 | 0.33 | 1.48 |
| ZSM-5 #3 pure zeolite (5 days crystallization) | 680 | 34.8 | 23.93 | 0.81 | 0.45 | 0.42 | 1.68 |
| Average of the 3 ZSM-5's prepd from TEOS | | 34.2 | 23.92 | 0.80 | 0.48 | 0.40 | 1.68 |

*TEOS = tetraethyl orthosilicate

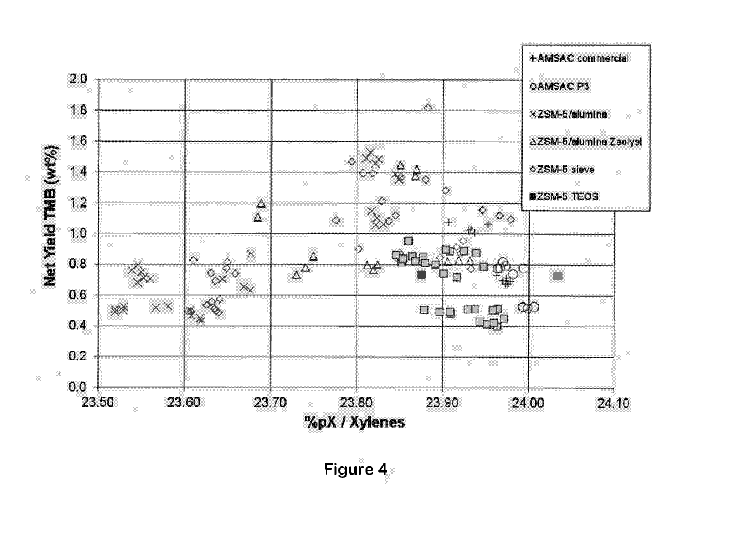

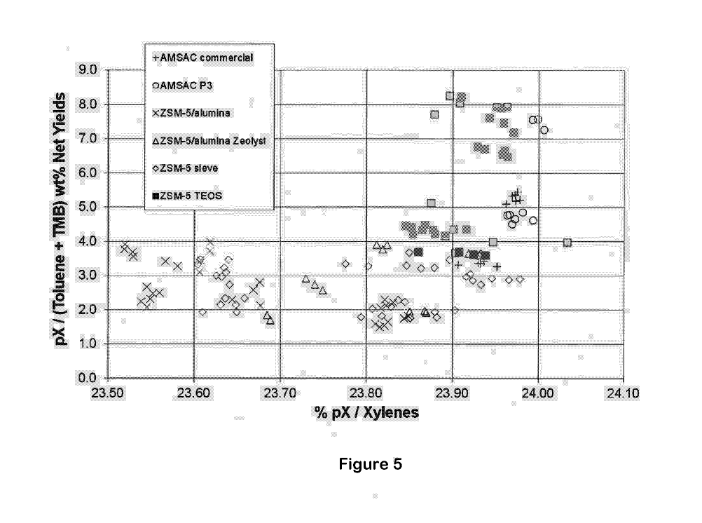

The catalysts were compared over a narrow temperature range (650° F. or 680° F.) and at similar ethylbenzene conversions (32-38%). The data in the fourth column indicate the extent of xylene isomerization catalyzed by the particular MFI, where the thermodynamic maximum % p-xylene isomer is about 24.1%. The results indicate that the MFI catalysts prepared from TEOS produced significantly lower yields of undesired trans-methylation products (toluene, trimethylbenzene (TMB), and methylethylbenzene (MEB)) than the commercial MFI catalysts (as shown in FIGS. 4 and 5). If fact, yields of these undesired products were typically about one-half those of the commercial MFI aluminosilicate catalysts. In addition, the MFI catalysts prepared from TEOS were highly active for xylene isomerization, yielding at least 23.9% p-xylene isomer in the effluent xylenes.

What is claimed is:

1. A method of increasing the proportion of p-xylene (pX) in a hydrocarbon-containing feed stream comprising xylene isomers, said method comprising:
contacting the hydrocarbon-containing feed stream with an isomerization catalyst under conditions suitable to yield a stream enriched in p-xylene with respect to the hydrocarbon-containing feed stream, wherein
the isomerization catalyst comprises a WI aluminosilicate molecular sieve prepared using a silicon source comprising a compound of the formula, Si(OR₁)(OR₂)(OR₃)(OR₄), wherein R₁R₂R₃R₄ is each independently a C₁₋₁₀alkyl or aryl.

2. The method of claim 1, further comprising recovering byproducts from the pX enriched stream.

3. The method of claim 2, wherein the byproducts contain 1.5 wt. % or less net toluene byproduct.

4. The method of claim 2, wherein the byproducts contain 3.5 wt. % or less net C₉-byproducts.

5. The method of claim 1, wherein the pX enriched stream contains less than 0.7 wt. % net trimethylbenzene byproduct.

6. The method of claim 1, wherein the pX enriched stream contains less than 1.0 wt % net toluene.

7. The method of claim 1, wherein the pX enriched stream contains less than 0.5 wt. % net trimethylbenzene byproduct.

8. A method of increasing the proportion of p-xylene (pX) in a hydrocarbon-containing feed stream comprising xylene isomers, said method comprising:
contacting the hydrocarbon-containing feed stream with an isomerization catalyst under conditions suitable to yield a stream enriched in p-xylene with respect to the hydrocarbon-containing feed stream, wherein
the isomerization catalyst comprises a MFI aluminosilicate molecular sieve; and
the pX enriched stream contains at least 23.5 wt. % pX/X and less than 1.5 wt. % net toluene byproduct.

9. A method of increasing the proportion of p-xylene (pX) in a hydrocarbon-containing feed stream comprising xylene isomers, said method comprising:
contacting the hydrocarbon-containing feed stream with an isomerization catalyst under conditions suitable to yield a stream enriched in p-xylene with respect to the hydrocarbon-containing feed stream, wherein
the isomerization catalyst comprises a MFI aluminosilicate molecular sieve; and
the pX enriched stream contains at least 23.8 wt. % pX/X and less than 0.6 wt. % net trimethylbenzene byproduct.

10. A method of increasing the proportion of p-xylene (pX) in a hydrocarbon-containing feed stream comprising xylene isomers, said method comprising:
contacting the hydrocarbon-containing feed stream with an isomerization catalyst under conditions suitable to yield a stream enriched in p-xylene with respect to the hydrocarbon-containing, feed stream, wherein
the isomerization catalyst comprises a MFI aluminosilicate molecular sieve; and
the pX enriched stream contains at least 23.5 wt. % pX/X and a ratio of pX/X to the sum of net wt. % trimethylbenzene byproduct and net wt. % toluene byproduct of greater than 4.0.

11. The method of claim 1, wherein the hydrocarbon-containing feed stream comprises at least 80 wt. % xylene isomers and pX/X of less than 12 wt. %.

12. The method of claim 1, wherein the hydrocarbon-containing feed stream is contacted with the isomerization catalyst in the presence of hydrogen.

13. The method of claim 1, further comprising recovering a pX product from the pX enriched stream, thereby forming a pX-lean stream.

14. The method of claim 13, wherein the pX-lean stream is recycled for use as the hydrocarbon-containing feed stream.

15. The method of claim 1, further comprising forming a combination stream by combining a make-up feed stream comprising xylene isomers with the pX enriched stream.

16. The method of claim 15, further comprising recovering a pX product from the combination stream, thereby forming a pX-lean stream for use as a hydrocarbon-containing feed stream.

17. The method of claim 15, further comprising recovering byproducts from the combination stream.

18. The method of claim 1, further comprising contacting the hydrocarbon-containing feed stream with an ethylbenzene (EB) conversion catalyst under conditions suitable to reduce the EB content of the hydrocarbon-containing feed stream.

19. The method of claim 18, wherein the hydrocarbon-containing feed stream is contacted with the EB conversion catalyst prior to being contacted with the isomerization catalyst.

20. The method of claim 18, wherein the hydrocarbon-containing feed stream is contacted with the EB conversion catalyst and the isomerization catalyst in a single reaction zone.

21. The method of claim 18, wherein the EB conversion catalyst comprises a MFI aluminosilicate molecular sieve.

22. The method of claim 1, wherein the isomerization catalyst and/or the EB conversion catalyst further comprises a support.

23. The method of claim 22, wherein the support comprises alumina, silica, and combinations thereof.

24. The method of claim 23, wherein the isomerization catalyst comprises 1-99 wt. % of the aluminosilicate molecular sieve.

25. A catalyst system for enriching a xylene isomers feed in p-xylene comprising a first bed comprising an ethylbenzene (EB) conversion catalyst and a second bed comprising an isomerization catalyst that is a MFI aluminosilicate catalyst prepared using a silicon source comprising a compound of the formula, Si(OR₁)(OR₂)(OR₃)(OR₄), wherein R₁R₂R₃R₄ is each independently a C₁₋₁₀alkyl or aryl.

26. The catalyst system of claim 25, wherein the EB conversion catalyst comprises an MFI aluminosilicate molecular sieve.

27. The catalyst system of claim 25, wherein the isomerization catalyst is prepared by:
combining an aluminum source and a template with the silicon source to form a reaction mixture;
removing byproducts from the reaction mixture to yield a concentrated reaction mixture;
heating the concentrated reaction mixture at a temperature and for a period of time suitable to yield a product mixture comprising a solid in an autoclave at autogeneous pressure;
isolating the solid from the product mixture; and
calcining the solid to yield the isomerization catalyst.

28. The catalyst system of claim 27, wherein the aluminum source comprises an aluminum C₁₋₁₀alkanoate or an aluminum C₁₋₁₀alkoxide.

29. The catalyst system of claim 27, wherein the template comprises tetrapropylammonium hydroxide or tetrapropylammonium bromide.

30. The catalyst system of claim 27, wherein the silicon source comprises tetra(alkyl) orthosilicate.

31. The catalyst system of claim 27, wherein the calcining is at a temperature between 480° C. and 600° C.

32. The catalyst system of any one of claims 25, wherein the isomerization catalyst further comprises a support.

33. The catalyst system of claim 32, wherein the support comprises alumina, silica, or combinations thereof.

34. The catalyst system of claim 33, wherein the isomerization catalyst comprises 1-99 wt. % MFI aluminosilicate molecular sieve.

35. The catalyst system of any one of claims 25, wherein the first bed is disposed over the second bed.

36. The catalyst system of claim 35, wherein a guard bed comprising a hydrogenation catalyst component and alumina is disposed over the first bed.

37. The catalyst system of claim 35, wherein a guard bed comprising a hydrogenation catalyst component and alumina is disposed between the first bed and the second bed.

38. A xylene isomerization reactor comprising a reaction zone containing a catalyst system of claim 25.

39. The method of claim 1, wherein the isomerization catalyst further comprises a hydrogenation catalyst component.

40. The catalyst system of claim 25, further comprising a hydrogenation catalyst component.
